(19) United States
(12) Patent Application Publication
(10) Pub. No.: US 2013/0103066 A1
(43) Pub. Date: Apr. 25, 2013

(54) Sonotrode

(71) Applicant: Soring GmbH, Quickborn (DE)

(72) Inventor: Abtin Jamshidi Rad, Hamburg (DE)

(73) Assignee: Soring GmbH, Quickborn (DE)

(21) Appl. No.: 13/654,687

(22) Filed: Oct. 18, 2012

(30) Foreign Application Priority Data
Oct. 19, 2011 (DE) 10 2011 084 792.8

(51) Int. Cl. A61B 17/32

(52) U.S. Cl. 606/169

(57) ABSTRACT

A sonotrode for an ultrasonic surgical instrument. The sonotrode comprises an instrument head which is equipped with a cutting apparatus that extends in the longitudinal direction. The instrument head is provided with a plurality of through-holes, which extend through the instrument head in the transverse direction. The through-holes impart an elastic inherent mobility to the instrument head, by means of which the removal of broken-up bone material is promoted.

### BACKGROUND

The invention relates to a sonotrode for an ultrasonic surgical instrument. The sonotrode comprises an instrument head which is equipped with a cutting apparatus that extends in the longitudinal direction.

Ultrasonic surgical instruments are used for severing tissue of a patient in surgical operations. An ultrasound transducer creates a high-frequency mechanical vibration. The sonotrode is connected to the ultrasound transducer and made to vibrate by the ultrasound transducer. If the cutting apparatus of the sonotrode is brought into contact with the tissue, the tissue is severed by the high-frequency vibration.

Ultrasonic surgical instruments of this type have been used successfully for a long time for cutting soft tissue. It is desirable to use the advantages of ultrasonic surgical instruments also when treating and severing bones. The high-frequency vibrations of the instrument head are in actual fact suitable for breaking up the structure of the bone material. However, the broken-up bone material prevents the instrument head from penetrating further into the bone and creating a deeper incision. Therefore, the known sonotrodes are less well-suited to treating and severing bone material.

### SUMMARY

A sonotrode which is well-suited to treating and severing bone material is presented.

The instrument head is provided with a plurality of through-holes, which extend through the instrument head in the transverse direction.

The mechanical properties of the instrument head are influenced in a targeted fashion by the through-holes. With respect to low frequency forces, the instrument head has a similar rigidity as conventional sonotrodes. The surgeon can therefore, in a familiar fashion, apply pressure on the work region or carry out a manual sawing movement. By contrast, with respect to the high-frequency vibrations of the ultrasound transducer, an elastic intrinsic mobility is imparted on the instrument head with the through-holes. By virtue of the through-holes being aligned transversely with respect to the longitudinal direction of the instrument head, the instrument head undergoes an intrinsic movement which is superposed on the high-frequency vibration driven by the ultrasound transducer. This combined movement leads firstly to new bone material continuously being broken up in terms of its structure and secondly to the broken-up bone material being removed from the work region. As a result of removing the broken-up bone material a space is created into which the instrument head can penetrate in order to break up further bone material. As a result of this, the instrument head can penetrate ever further into the bone and create an incision. Moreover, the temperature rise is kept in check as a result of the rapid removal of the broken-up bone material.

The through-holes should be arranged such that the elastic intrinsic mobility of the instrument head is pronounced. To this end it is advantageous if the central axes of the through-holes are aligned parallel to one another. Moreover, the through-holes can be arranged behind one another in the longitudinal direction of the instrument head. Should the central axes of the through-holes be arranged parallel to one another, this means that the central axes lie in a plane which is spanned by the longitudinal direction and the transverse direction of the instrument head.

When observed in the cross section (i.e., in a plane aligned perpendicularly to the central axis of the through-hole), the through-holes can have an elongate form. Here, the largest cross-sectional extent is preferably at least double, more preferably at least three times, the smallest cross-sectional extent. The largest cross-sectional extent is preferably so long that it can cover at least 50% of the height of the instrument head.

The largest cross-sectional extent of the through-hole can include an angle with the longitudinal direction of the instrument head. An advantage of this is that the forces in the instrument head are deflected and the elastic movement thereby obtains a different direction than the high-frequency vibration. The broken-up bone material is then removed in a particularly effective fashion. The angle between the largest cross-sectional extent of the through-hole and the longitudinal direction of the instrument head is preferably between 30° and 80°, more preferably between 50° and 70°. The cutting surface-side end of the through-hole with an elongate cross section is preferably situated closer to the rear (proximal) end of the instrument head than the opposing end. The elastic mobility of the instrument head then leads to a nodding movement, by means of which the broken-up bone material is removed in a particularly effective fashion.

It is advantageous for the elastic mobility of the instrument head if the through-holes are situated closely together. By way of example, the through-holes can be arranged such that the distance of the central axes of two adjacent through-holes is at most double the cross-sectional extent of the through-holes in the relevant direction. If the through-holes have an elongate cross section, respectively one material web remains between two through-holes. This material web is preferably at least twice as long as it is wide. The elastic mobility of the instrument head obtained by the through-holes is moreover advantageous in that squeaking noises are reduced compared to intrinsically rigid sonotrodes.

The cutting apparatus of the instrument head preferably has an areal extent. In the case of such a cutting surface, the risk of inadvertent injury to the tissue is lower than in the case of a sharp cutting edge. In an advantageous embodiment, the cutting surface is aligned substantially parallel to a surface spanned by the central axes of the through-holes. The amplitude and frequency of the high-frequency vibration are preferably set such that the cutting effect is only small when simply contacting the bone. The cutting effect is only maximised by a superposition of the high-frequency vibration with a manual sawing movement carried out by the surgeon.

The cutting surface can be provided with a plurality of cut-outs, which extend in the transverse direction of the instrument head. A plurality of these cut-outs can be arranged behind one another in the longitudinal direction of the instrument head. If the high-frequency vibration of the sonotrode is a movement in the longitudinal direction of the instrument head, the cut-outs are aligned transversely with respect to the movement direction. By way of example, the cut-outs can have an approximately semi-circular cross section. The bone material can be treated effectively with the structure formed by the cut-outs.

The cut-outs taper off from the edge of the cutting surface in the direction of the centre in an advantageous embodiment such that the cut-outs have an hourglass-like form. When viewed in the longitudinal direction of the instrument head, the distance between two adjacent cut-outs is then greater in the centre than at the edge of the cutting surface. Thus, there is a rhombus-like surface section in the centre of the cutting surface between two cut-outs, which surface section is preferably substantially planar. This surface section acts as contact surface, which rests areally on the bone to be treated. At the edge of the cutting surface, the cut-outs—as viewed in the longitudinal direction of the instrument head—preferably directly adjoin one another.

This design of the cut-outs is advantageous in that the bone can be treated effectively by the edges at the transition between the contact surfaces and the cut-outs. In the process, the broken-up bone material is routed to the outside by the obliquely aligned edges. Secondly, there is no damage to soft tissue which inadvertently comes into contact with the sonotrode. This is because if the frequency and amplitude of the high-frequency vibration are suitably selected, the soft tissue itself is made to vibrate by the areally resting contact surfaces instead of being severed. When the sonotrode rests against soft tissue, the vibration property of the instrument also changes, and so the surgeon obtains direct feedback if work is no longer performed on the bone. It is for this reason that the instrument can also be used for work in the direct vicinity of e.g., nerves or blood vessels. This embodiment of the cut-outs has a self-contained inventive content, even without the instrument head being provided with through-holes.

The sonotrode regularly comprises a shaft connected to the instrument head. A connection to the ultrasound transducer can be established via the other end of the shaft. The shaft is preferably aligned in the longitudinal direction of the instrument head. The shaft forms the proximal end of the sonotrode; the distal end is situated opposite thereto. In respect of the instrument head, the terms proximal and distal are used correspondingly.

The extent of the instrument head in the longitudinal direction can lie between 5 mm and 15 mm, preferably between 8 mm and 10 mm. The extent of the instrument head in the transverse direction can for example lie between 1 mm and 5 mm. The extent in the longitudinal direction is regularly greater than the extent in the transverse direction. The height perpendicular to the longitudinal direction and the transverse direction can likewise lie between 1 mm and 5 mm.

A surgical instrument with an ultrasound transducer and a sonotrode is connected to the ultrasound transducer. The ultrasound transducer may comprise a piezoelectric element, by means of which a high-frequency AC voltage is converted into a corresponding mechanical vibration. By way of example, the frequency of the vibration can lie between 20 kHz and 40 kHz.

The ultrasonic surgical instrument is regularly equipped with a line by means of which a rinsing liquid can be supplied to the operating field. The movement of the sonotrode forms rinsing liquid vortices in the surroundings of the instrument head, which also enter the through-holes and as a result bring about an effective cooling of the instrument head.

### BRIEF DESCRIPTION OF THE DRAWINGS

In the following text, the invention will be explained in an exemplary fashion on the basis of an advantageous embodiment, with reference being made to the attached drawings. In detail:

FIG. 1 shows a lateral view of an ultrasonic surgical instrument;

FIG. 2 shows a lateral view of a sonotrode;

FIG. 3 shows the instrument head of the sonotrode from FIG. 2 in a magnified illustration; and

FIG. 4 shows a view from below of the instrument head from FIG. 2.

### DETAILED DESCRIPTION

At its rear end, an ultrasonic surgical instrument in FIG. 1 comprises a handle 14, by means of which the surgeon can guide the instrument. Arranged in the interior of the instrument there is an ultrasound transducer (not visible in FIG. 1), which obtains an electrical AC voltage signal as input signal from a signal generator (likewise not illustrated in FIG. 1). By way of example, the AC frequency can lie between 20 kHz and 40 kHz. The ultrasound transducer comprises a piezoelectric element, by means of which the electrical signal is converted into a mechanical vibration which is aligned along the longitudinal direction of the instrument. The mechanical vibration is transmitted to a sonotrode 16, which comprises a shaft 19 with an instrument head 17 at the front end. The surface of the instrument head 17 illustrated toward the bottom in FIG. 1 serves as cutting surface 18. If the vibrating cutting surface 18 of the vibrating instrument head 17 is led to a bone, the bone material is severed. The ultrasonic surgical instrument moreover comprises a line 12 (visible in FIG. 1) in order to be able to supply a rinsing liquid to the operating field.

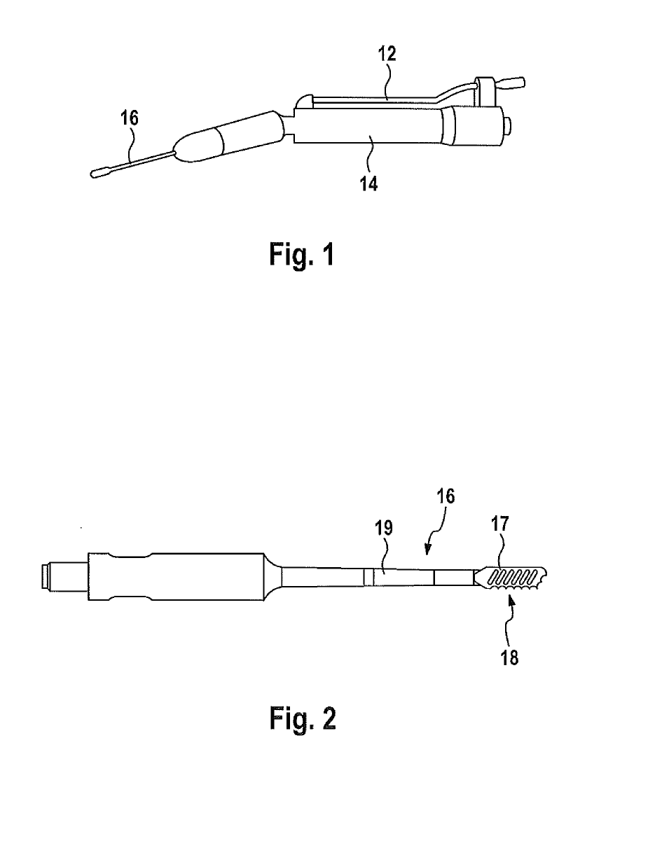

The instrument head 17 of the sonotrode 16 from FIG. 1 is shown in a magnified illustration in FIG. 2. According thereto, the instrument head 17 is provided with six through-holes 20, which extend through the instrument head 17 in the transverse direction (perpendicular to the plane of the image in FIG. 2). The through-holes 20 are channels which completely pass through the instrument head. When observed in the cross section (in the image plane in FIG. 2), the through-holes 20 have an elongate form, with the largest cross-sectional extent running obliquely from bottom left to top right. The largest cross-sectional extent includes an angle a of approximately 60° with the longitudinal direction of the sonotrode.

The central axes of the through-holes 20 are arranged parallel to one another and all lie in a plane which is spanned by the longitudinal axis of the sonotrode 16 and the transverse direction. The distance of the central axes is such that the webs 21 respectively remaining between two through-holes 20 are thin compared to the largest cross-sectional extent of the through-holes 20.

The instrument head 17 is intrinsically elastic as a result of the through-holes 20, and so it acts like a spring element with respect to the high-frequency vibrations of the sonotrode 16. If the sonotrode 16 vibrates in the longitudinal direction, there is an elastic intrinsic movement, in the form of a nodding motion, in the instrument head 17. The nodding motion substantially occurs in the image plane of FIG. 1.

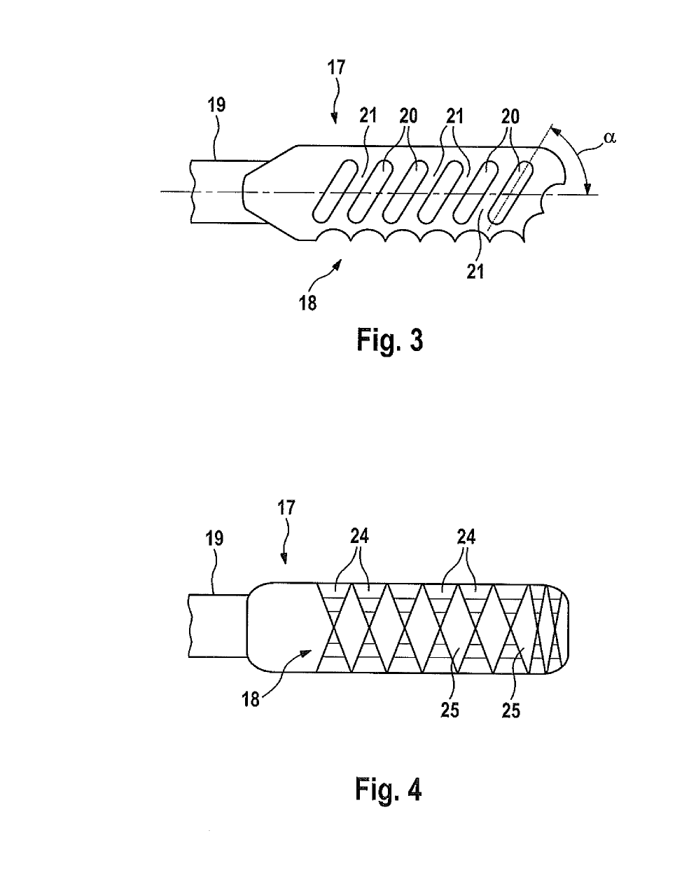

FIG. 3 shows a view from below on the instrument head 17 from FIG. 2 and hence a plan view on the cutting surface 18 of the instrument head 17. A plurality of cut-outs 24 are formed in the cutting surface 18. The cut-outs 24 taper off from the edge towards the centre from both sides, and so they have an hourglass-like form. The cut-outs 24 have an approximately semi-circular cross section. Two respectively adjacent cut-outs directly adjoin one another on the edge. In the centre, the cut-outs 24 have a distance from one another which is filled by a planar contact surface 25. The contact surfaces 25 have the form of a rhombus in FIG. 3.

When the ultrasonic surgical instrument is used, the surgeon initially puts the ultrasound transducer into operation such that the sonotrode 16 is made to vibrate in the longitudinal direction. The surgeon then moves the instrument such that the instrument head 17 performs a manual sawing motion with the cutting surface 18 on the bone to be severed. The bone material is severed by the superposition of the manual sawing motion with the high-frequency vibration. In this case, it is sufficient if a light pressure of approximately 100 g is exerted on the instrument. The cutting effect emerges in particular as a result of the edges between the contact surfaces 25 and the cut-outs 24. The structure of the bone material is broken down, and so the instrument head 17 can penetrate the bone material. A cut is created, the width of which corresponds to the width of the instrument head.

The bone material, the structure of which has been broken down, is removed from the work region as a result of the nodding movement intrinsically carried out by the instrument head 17 while the sonotrode 16 vibrates. Thus, the instrument head 17 is not prevented from penetrating deeper into the bone material. The removal of the broken-down bone material is furthermore promoted by the design of the cut-outs 24, which comprises obliquely outwardly facing surfaces.

There is only a small cutting effect if the instrument head comes into contact with soft tissue rather than with bone material. As a result of the large contact surfaces 25 between the cut-outs 24, the cutting surface 18 rests substantially areally on the soft tissue. The soft tissue is therefore not severed but merely made to vibrate. The instrument therefore provides protection against the instrument inadvertently severing soft tissue rather than the bone as a result of careless guidance.

1. Sonotrode for an ultrasonic surgical instrument, comprising an instrument head which is equipped with a cutting apparatus that extends in a longitudinal direction, wherein the instrument head is provided with a plurality of through-holes, which extend through the instrument head in the transverse direction, wherein the through-holes have an elongate cross-section and wherein the largest cross-sectional extent of the through-hole includes an angle between 30° and 80° with the longitudinal direction of the instrument head.

2. Sonotrode according to claim 1, characterized in that the central axes of the through-holes are arranged parallel to one another.

3. Sonotrode according to claim 1, characterized in that the through-holes are arranged behind one another in the longitudinal direction of the instrument head.

4. Sonotrode according to claim 1, characterized in that the angle between the largest cross-sectional extent of the through-hole and the longitudinal direction of the instrument head lies between 50° and 70°.

5. Sonotrode according to claim 1, characterized in that the end of the through-hole facing the cutting apparatus is closer to the proximal end of the instrument head than the end of the through-hole distant from the cutting apparatus.

6. Sonotrode according to claim 1, characterized in that the cutting apparatus is embodied as cutting surface.

7. Sonotrode according to claim 6, characterized in that the cutting surface is aligned substantially parallel to a plane spanned by the central axes of the through-holes.

8. Sonotrode according to claim 1, characterized in that the cutting surface is provided with a plurality of cut-outs, which extend in the transverse direction of the instrument head.

9. Sonotrode according to claim 8, characterized in that the cut-outs taper off from the edge of the instrument head in the direction of the centre.

10. Surgical instrument with an ultrasound transducer and a sonotrode connected to the ultrasound transducer, characterized in that the sonotrode is embodied according to claim 1.

11. Surgical instrument with an ultrasound transducer and a sonotrode connected to the ultrasound transducer, characterized in that the sonotrode is embodied according to claim 2.

12. Surgical instrument with an ultrasound transducer and a sonotrode connected to the ultrasound transducer, characterized in that the sonotrode is embodied according to claim 3.

13. Surgical instrument with an ultrasound transducer and a sonotrode connected to the ultrasound transducer, characterized in that the sonotrode is embodied according to claim 4.

14. Surgical instrument with an ultrasound transducer and a sonotrode connected to the ultrasound transducer, characterized in that the sonotrode is embodied according to claim 5.

15. Surgical instrument with an ultrasound transducer and a sonotrode connected to the ultrasound transducer, characterized in that the sonotrode is embodied according to claim 6.

16. Surgical instrument with an ultrasound transducer and a sonotrode connected to the ultrasound transducer, characterized in that the sonotrode is embodied according to claim 7.

17. Surgical instrument with an ultrasound transducer and a sonotrode connected to the ultrasound transducer, characterized in that the sonotrode is embodied according to claim 8.

18. Surgical instrument with an ultrasound transducer and a sonotrode connected to the ultrasound transducer, characterized in that the sonotrode is embodied according to claim 9.

19. Sonotrode according to claim 2, characterized in that the through-holes are arranged behind one another in the longitudinal direction of the instrument head.

20. Sonotrode according to claim 2, characterized in that the angle between the largest cross-sectional extent of the through-hole and the longitudinal direction of the instrument head lies between 50° and 70°.
